(19) United States
(12) Patent Application Publication
(10) Pub. No.: US 2024/0058393 A1
(43) Pub. Date: Feb. 22, 2024

(54) COMPOSITION COMPRISING MICROCOCCUS LUTEUS-DERIVED EXTRACELLULAR VESICLE FOR PREVENTION OR TREATMENT OF METABOLIC DISEASE

(71) Applicant: MD HEALTHCARE INC., Seoul (KR)

(72) Inventor: Yoon-Keun KIM, Paju-Si, Gyeonggi-do (KR)

(73) Assignee: MD HEALTHCARE INC., Seoul (KR)

(21) Appl. No.: 18/259,668

(22) PCT Filed: Nov. 15, 2021
(86) PCT No.: PCT/KR2021/016645

§ 371 Date: Jun. 28, 2023

(30) Foreign Application Priority Data
Dec. 28, 2020 (KR) 10-2020-0184337
Nov. 12, 2021 (KR) 10-2021-0156026

(51) Int. Cl. A61K 35/74; A61K 9/127; A61P 13/12; A61P 1/16

(52) U.S. Cl. CPC A61K 35/74; A61K 9/127; A61P 13/12; A61P 1/16

(57) ABSTRACT

Provided is Micrococcus luteus-derived extracellular vesicles and a use thereof and, more particularly, to a composition including Micrococcus luteus-derived extracellular vesicles as an active ingredient for alleviation, prevention, or treatment of metabolic diseases, wherein the composition can effectively treat metabolic diseases occurring as a result of a metabolism disorder, including: metabolic cardiovascular diseases such as metabolic syndrome, arteriosclerosis, stroke, and cardiac failure; metabolic liver diseases such as non-alcoholic steatohepatitis and hepatocirrhosis; metabolic renal diseases such as diabetic nephropathy, hypertensive nephropathy, and renal failure; and metabolic musculoskeletal disorders such as gout, sarcopenia, and osteoporosis.

### TECHNICAL FIELD

The present invention relates to Micrococcus luteus-derived extracellular vesicles and a use thereof, and more particularly, to a composition and the like for preventing or treating metabolic diseases, comprising extracellular vesicles derived from Micrococcus luteus as an active ingredient.

This application claims priority to and the benefit of Korean Patent Application Nos. 10-2020-0184337 and 10-2021-0156026 filed in the Korean Intellectual Property Office on Dec. 28, 2020 and Nov. 12, 2021, respectively, and all the contents disclosed in the specification and drawings of the applications are incorporated in this application.

### BACKGROUND ART

As the 21st century begins, the importance of acute infectious diseases that were recognized as epidemics in the past has decreased, whereas metabolism disorders occurring in the major organs of our body and chronic inflammatory diseases caused by the metabolism disorders have changed the pattern of diseases to become a major disease that reduces the quality of life and determines a human's life expectancy. Such changes in disease patterns are closely related to changes in diet, and in the past, acute infectious diseases were the main cause of death in the era of nutritional deficiency, but recently, obesity caused by overnutrition and related metabolic diseases have become major problems.

As a result of metabolic disorders caused by overnutrition, fat accumulates not only in adipose tissue but also in blood vessels, the heart, liver, kidneys, muscle, and the like, resulting in lipotoxicity, which causes metabolic diseases. That is, carbohydrate or lipid metabolism disorders increase blood lipids and fatty acids, and cause inflammatory responses due to fatty acids while increasing fatty acids in cells, and the inflammatory response causes cellular senescence and death, resulting in dysfunction in organs such as the cardiovascular system, liver, kidneys, and muscle. Metabolic diseases caused by such etiologies include metabolic cardiovascular diseases such as arteriosclerosis, metabolic syndrome, and cardiac failure; metabolic liver diseases such as non-alcoholic steatohepatitis and hepatocirrhosis; metabolic renal diseases such as chronic nephropathy and renal failure; and metabolic musculoskeletal disorders such as gout, sarcopenia, and osteoporosis.

Although normal cellular metabolism balances the production and use of lipids, fatty acids are converted into triacylglycerols or diacylglycerols such as ceramide or fatty acyl-CoA when more fatty acids are produced than are oxidized for energy production. Triacylglycerols do not induce inflammatory responses in cells, but diacylglycerols such as ceramide or fatty acyl-CoA induce inflammatory responses in cells, and thus induce cellular senescence and death. In addition, free fatty acids induce inflammatory responses through toll-like receptor 4 (TLR-4) which is a pattern recognition receptor (PRR), resulting in cell death.

Energy metabolism creates the energy required to carry out functions of cells and produces various materials, thereby synthesizing proteins and lipids in the endoplasmic reticulum (ER) through ATP produced by mitochondria and supplying the proteins and lipids to a required region. Cells face various stresses from the moment they are generated, biological, chemical, physical, and psychological stresses induce endoplasmic reticulum (ER) stress, mitochondrial dysfunction, and lysosomal damage, and the like in cells, which activates an NLR family pyrin domain containing 3 inflammasome (NLRP3 inflammasome) to induce inflammation and cell death, causing various diseases.

Immunity is a defense mechanism of cells against biological, chemical, physical, and mental stress, and occurs through innate immunity and adaptive immunity. Recently, in relation to the pathogenesis of inflammatory diseases caused by metabolic disorders, cytoplasmic fatty acids, uric acid, and other metabolites are recognized as danger signals by a nucleotide-binding oligomerization domain, leucine rich repeat and pyrin domain containing (NLRP), which is a pattern recognition receptor present in the cytoplasm, and the fact that among them, the NLRP3 protein forms an inflammasome to cause various metabolic diseases has become known.

It is known that the number of microorganisms that coexist in the human body reaches 100 trillion, which is about 10-fold larger than that of human cells, and the number of genes of microorganisms is 100-fold larger than that of humans. A microbiota or microbiome refers to a microbial community including bacteria, archaea and eukarya present in a given habitat.

Bacteria that coexist in our bodies and bacteria that exist in the surrounding environment secrete nanometer-sized vesicles to exchange information such as genes, low molecular compounds, and proteins with other cells. The mucosa forms a physical defense membrane through which particles having a size of 200 nanometers (nm) or more cannot pass, so that bacteria coexisting in the mucosa cannot pass through the mucosa, but bacteria-derived vesicles have a size of 200 nanometers or less, and thus relatively freely pass through epithelial cells via the mucosa to be absorbed in our bodies. As described above, although bacteria-derived vesicles are secreted from bacteria, they differ from bacteria in terms of their constituents, absorption rate in the body, and risk of side effects, and therefore, the use of bacteria-derived vesicles is completely different from that of living cells or has a significant effect.

Locally secreted bacteria-derived vesicles are absorbed through the epithelial cells of the mucosa to induce a local inflammatory response, and vesicles that have passed through the epithelial cells are systemically absorbed through the lymphatic vessels to be distributed to respective organs, and regulate immune and inflammatory responses in the organs to which the vesicles are distributed. For example, vesicles derived from pathogenic gram-negative bacteria, such as Escherichia coli, are pathogenic nanoparticles that cause local colitis, are absorbed into vascular endothelial cells when absorbed by blood vessels to promote a systemic inflammatory response and blood coagulation by inducing an inflammatory response, and are also absorbed into muscle cells where insulin acts to cause insulin resistance and diabetes mellitus. In contrast, vesicles derived from beneficial bacteria may regulate a disease by regulating immune and metabolic dysfunctions caused by pathogenic vesicles.

Micrococcus luteus is a gram-positive bacterium belonging to the genus Micrococcus, and a bacterium that is widely distributed in nature such as water, dust, and soil. This bacterium is known to produce riboflavin when grown in toxic organic pollutants such as pyridine and absorb ultraviolet light through lutein pigment. In addition, it is known that the bacterium is also isolated from dairy products and beer, grows in dry or high-salt environments, and does not form spores, but survives at a refrigeration temperature in a refrigerator for a long period of time.

However, there has been no report in which vesicles derived from Micrococcus luteus are applied to the treatment of metabolic diseases to date.

### DISCLOSURE

### Technical Problem

As a result of intensive studies to solve the problems described above, the present inventors confirmed that when cells were treated with Micrococcus luteus-derived vesicles isolated from a culture solution in which Micrococcus luteus had been cultured, the secretion of inflammatory mediators by pathogenic causative factors was remarkably suppressed, and Micrococcus luteus-derived vesicles were able to efficiently suppress immune dysfunction induced by biological pathogenic factors. Further, the present inventors confirmed that Micrococcus luteus-derived vesicles also regulated immune function by suppressing the expression of NLRP3 protein, which is a pattern recognition receptor (PRR) associated with the pathogenesis of various diseases, and cell death was suppressed by increasing endothelial NO synthase (eNOS) signals. In addition, the present inventors confirmed that when a metabolic disease mouse model induced by a high-fat diet was treated with the vesicles, metabolic diseases were efficiently suppressed, thereby completing the present invention.

Thus, an object of the present invention is to provide a pharmaceutical composition for preventing or treating a metabolic disease, comprising Micrococcus luteus-derived vesicles as an active ingredient.

Another object of the present invention is to provide a food composition for preventing or alleviating a metabolic disease, comprising Micrococcus luteus-derived vesicles as an active ingredient.

Still another object of the present invention is to provide an inhalant composition for preventing or alleviating a metabolic disease, comprising Micrococcus luteus-derived vesicles as an active ingredient.

Yet another object of the present invention is to provide a composition for delivering a therapeutic drug for a liver disease or renal disease, comprising Micrococcus luteus-derived vesicles as an active ingredient.

However, a technical problem to be achieved by the present invention is not limited to the aforementioned problems, and the other problems that are not mentioned may be clearly understood by a person skilled in the art from the following description.

### Technical Solution

To achieve the object of the present invention as described above, the present invention provides a pharmaceutical composition for preventing or treating a metabolic disease, comprising Micrococcus luteus-derived vesicles as an active ingredient.

In addition, the present invention provides a food composition for preventing or alleviating a metabolic disease, comprising Micrococcus luteus-derived vesicles as an active ingredient.

In addition, the present invention provides an inhalant composition for preventing or alleviating a metabolic disease, comprising Micrococcus luteus-derived vesicles as an active ingredient.

Furthermore, the present invention provides a composition for delivering a therapeutic drug for a liver disease or renal disease, comprising Micrococcus luteus-derived vesicles as an active ingredient.

In an exemplary embodiment of the present invention, the metabolic disease may be one or more selected from the group consisting of a metabolic cardiovascular disease, a metabolic liver disease, a metabolic renal disease, and a metabolic musculoskeletal disease, but is not limited thereto.

In another exemplary embodiment of the present invention, the metabolic cardiovascular disease may be one or more selected from the group consisting of hyperinsulinemia, dyslipidemia, arrhythmia, metabolic syndrome, arteriosclerosis, stroke, and cardiac failure, but is not limited thereto.

In still another exemplary embodiment of the present invention, the metabolic liver disease may be one or more selected from the group consisting of hepatic triglyceride accumulation, simple steatosis, non-alcoholic steatohepatitis, and hepatocirrhosis, but is not limited thereto.

In yet another exemplary embodiment of the present invention, the metabolic renal disease may be one or more selected from the group consisting of glomerulonephritis, diabetic nephropathy, hypertensive nephropathy, and chronic renal failure, but is not limited thereto.

In yet another exemplary embodiment of the present invention, the metabolic musculoskeletal disease may be one or more selected from the group consisting of cachexia, gout, sarcopenia, and osteoporosis, but is not limited thereto.

In yet another exemplary embodiment of the present invention, the metabolic disease may be a metabolic disease mediated by an NLR family pyrin domain containing 3 inflammasome (NLRP3 inflammasome), but is not limited thereto.

In yet another exemplary embodiment of the present invention, the composition may suppress the formation of an NLRP3 inflammasome, but is not limited thereto.

As still another exemplary embodiment of the present invention, the vesicles may have an average diameter of 10 to 200 nm, but the average diameter is not limited thereto.

As yet another exemplary embodiment of the present invention, the vesicles may be naturally secreted or artificially produced from Micrococcus luteus, but are not limited thereto.

Further, the present invention provides a method for preventing or treating a metabolic disease, the method comprising administering a composition comprising Micrococcus luteus-derived vesicles as an active ingredient to a subject in need thereof.

In addition, the present invention provides a use of a composition comprising Micrococcus luteus-derived vesicles as an active ingredient for preventing or treating a metabolic disease.

Furthermore, the present invention provides a use of Micrococcus luteus-derived vesicles for preparing a drug for treating a metabolic disease.

Further, the present invention provides a method for delivering a drug for treating a liver disease or renal disease, the method comprising administering a composition comprising Micrococcus luteus-derived vesicles, which carry a drug for treating a liver disease or renal disease to be targeted, as an active ingredient to a subject in need thereof.

In addition, the present invention provides a use of a composition comprising Micrococcus luteus-derived vesicles as an active ingredient for delivering a therapeutic drug for a liver disease or renal disease.

### Advantageous Effects

The present inventors confirmed that when vesicles derived from Micrococcus luteus were orally administered, the vesicles were absorbed into blood vessels and distributed in organs such as liver and kidneys. Further, the present inventors confirmed that when epithelial cells and inflammatory cells were treated with the vesicles, the secretion of inflammatory mediators by biological causative factors was remarkably suppressed, and when cells were treated with the vesicles, NLRP3 protein expression and NF-kB signals induced by pathogenic factors were suppressed, and eNOS signals suppressed by pathogenic causative factors were increased. In addition, since it was confirmed that when the vesicles were administered to a mouse model of metabolic liver disease and kidney disease induced by a high-fat diet, hepatitis and renal dysfunction caused by metabolic disorders were efficiently suppressed, the Micrococcus luteus-derived vesicles according to the present invention can be usefully used not only for the development of pharmaceuticals or health functional foods for preventing, ameliorating symptoms or treating metabolic cardiovascular diseases such as arteriosclerosis, metabolic syndrome, and cardiac failure; metabolic liver diseases such as non-alcoholic steatohepatitis and hepatocirrhosis; metabolic renal diseases such as chronic nephropathy and renal failure; and metabolic musculoskeletal disorders such as gout, sarcopenia, and osteoporosis, but also as a drug delivery system for treating a liver disease or renal disease.

### DESCRIPTION OF DRAWINGS

FIG. 1 is a view illustrating the intensity of fluorescence in each organ by orally administering fluorescently labeled Micrococcus luteus-derived vesicles to mice and then removing each organ over time.

FIGS. 2A and 2B are views illustrating the distribution pattern of vesicles in the liver (a) and kidneys (b) over time after orally administering fluorescently labeled Micrococcus luteus-derived vesicles to mice.

FIG. 3 is a view illustrating an experimental protocol for evaluating the effect of suppressing the secretion of an inflammatory mediator by an E. coli-derived vesicle (E. coli EV), which is an inflammatory causative factor, by administering a Micrococcus luteus-derived vesicle (MDH-101 EV) or a positive control drug dexamethasone to epithelial cells.

FIGS. 4A and 4B are views illustrating the dose dependence (A) for suppressing the secretion of an inflammatory mediator IL-8 and experimental results (B) comparing the efficacy with a control drug dexamethasone, by administering a Micrococcus luteus-derived vesicle (MDH-101) to epithelial cells (*P<0.05, **P<0.01, ***P<0.001, n.s. means not significant, and hereinafter the same).

FIG. 5 is a view illustrating an experimental protocol for evaluating the effect of suppressing the secretion of an inflammatory mediator by an E. coli-derived vesicle (E. coli EV), which is an inflammatory causative factor, by administering a Micrococcus luteus-derived vesicle (M. luteus EV) to macrophages which are inflammatory cells.

FIGS. 6A and 6B are views confirming the secretion suppressing effect of a Micrococcus luteus-derived vesicle (M. luteus EV) on inflammatory mediators TNF-α (A) and IL-6 (B) due to an E. coli-derived vesicle (E. coli EV) in macrophages.

FIG. 7 is a view illustrating the effect of suppressing the secretion of an inflammatory mediator IL-1β by an E. coli-derived vesicle (E. coli EV) by administering Micrococcus luteus-derived vesicles (M. luteus EVs) to macrophages.

FIG. 8 is a view illustrating the results of confirming the expression patterns of immune function-regulatory proteins NLRP3, T-bet, and ROR-γt in tissues isolated from mice administered lipopolysaccharide (LPS), which is an inflammatory causative factor, in order to evaluate the effect of regulating immune function by a Micrococcus luteus-derived vesicle (MIEV).

FIG. 9 is a view illustrating the results of confirming the degree of activation of JNK and NF-kB (p65), which are signals associated with innate immunity, in tissues isolated from mice administered lipopolysaccharide (LPS) which is an inflammatory causative factor, in order to evaluate the effect of regulating innate immune function by a Micrococcus luteus-derived vesicle (MIEV).

FIG. 10 is a set of views confirming the number of type 3 innate lymphoid cells (ILC3) associated with the pathogenesis of a metabolic disease in tissues isolated from mice administered lipopolysaccharide (LPS) which is an inflammatory causative factor, in order to evaluate the effect of regulating the innate immune cell production by a Micrococcus luteus-derived vesicle (MIEV).

FIG. 11 is a view illustrating the results of confirming the influence on eNOS signals suppressed by an E. coli-derived vesicle, which is an inflammatory causative factor, by administering a Micrococcus luteus-derived vesicle (MDH-101) or a positive control drug dexamethasone to cells, in order to evaluate the effect of the Micrococcus luteus-derived vesicle on endothelial NO synthase (eNOS) signals, which are important for cell homeostasis.

FIG. 12 is an experimental protocol for evaluating the efficacy of a Micrococcus luteus-derived vesicle (EV) against metabolic diseases induced by a high-fat diet (60% HFD: high-fat diet containing 60% saturated fat, QD: once daily, PO: oral administration).

FIGS. 13A and 13B are views illustrating the results of measuring food intake (A) and body weight change (B) after orally administering a control drug metformin (Con+) or low-concentration (Case 1) or high-concentration (Case 2) of Micrococcus luteus-derived vesicles to a mouse model of metabolic disease induced by a high-fat diet.

FIGS. 14A and 14B are views illustrating the results of measuring plasma triglyceride (A) and plasma free acid concentration (B) after orally administering a control drug metformin (Con+) or a low-concentration (Case 1) or high-concentration (Case 2) of Micrococcus luteus-derived vesicles to a mouse model of metabolic disease induced by a high-fat diet.

FIG. 15 is a view illustrating the results of measuring plasma AST concentration and plasma ALT concentration after orally administering a control drug metformin (Con+) or a low-concentration (Case 1) or high-concentration (Case 2) of Micrococcus luteus-derived vesicles to a mouse model of metabolic disease induced by a high-fat diet.

FIG. 16 is a view illustrating an experimental protocol for evaluating the efficacy of Micrococcus luteus-derived vesicles on metabolic renal diseases caused by a high-fat diet.

FIGS. 17A and 17B are views illustrating the results of measuring plasma BUN concentration (A) and plasma creatinine concentration (B), which are renal function markers, after orally administering a Micrococcus luteus-derived vesicle (EV) to a mouse model of metabolic renal disease induced by a high-fat diet.

### [Best Model]

The present invention relates to vesicles derived from Micrococcus luteus and a use thereof.

Hereinafter, the present invention will be described in detail.

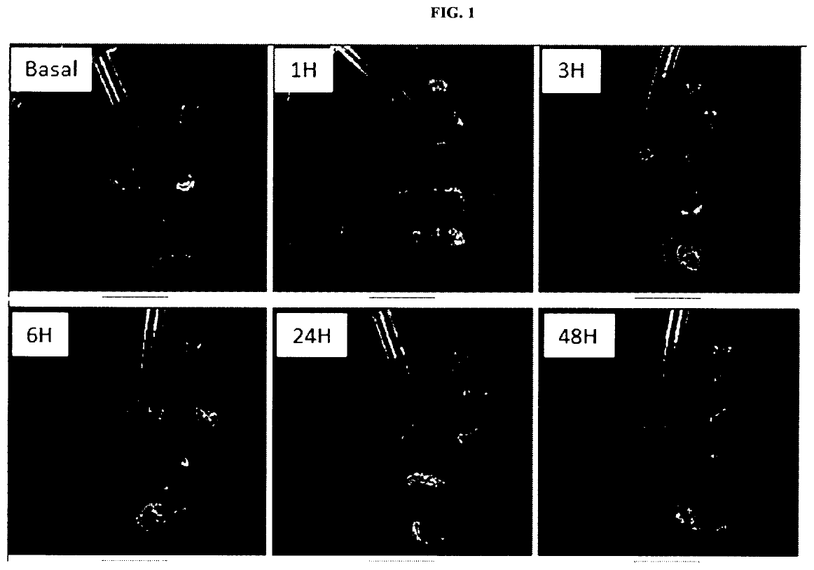

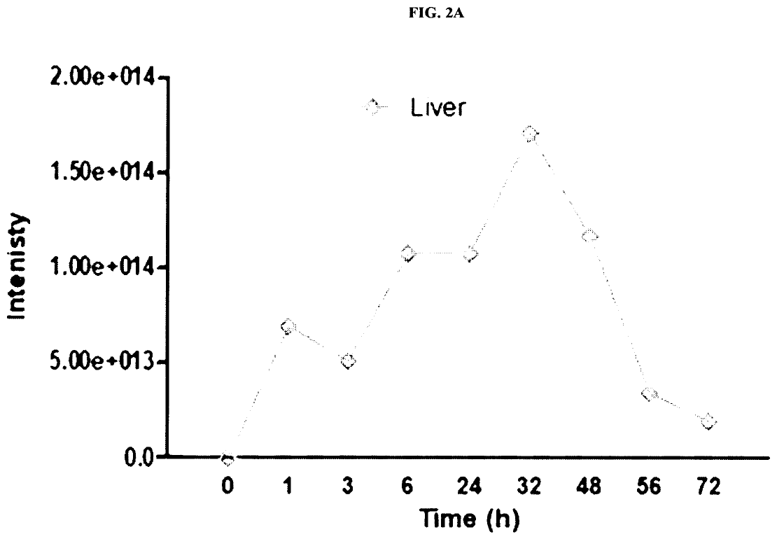

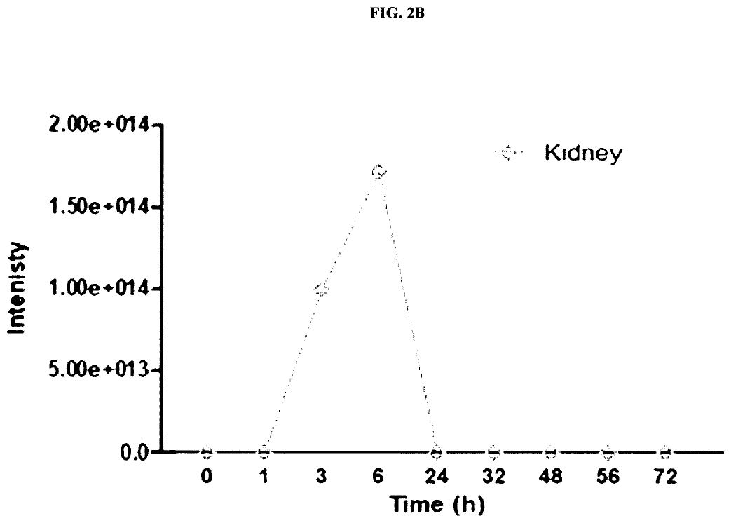

In an exemplary embodiment of the present invention, it was confirmed that when Micrococcus luteus-derived vesicles were orally administered, the vesicles were systemically absorbed and distributed to the liver and kidneys (see Example 2 and FIGS. 1 to 2B).

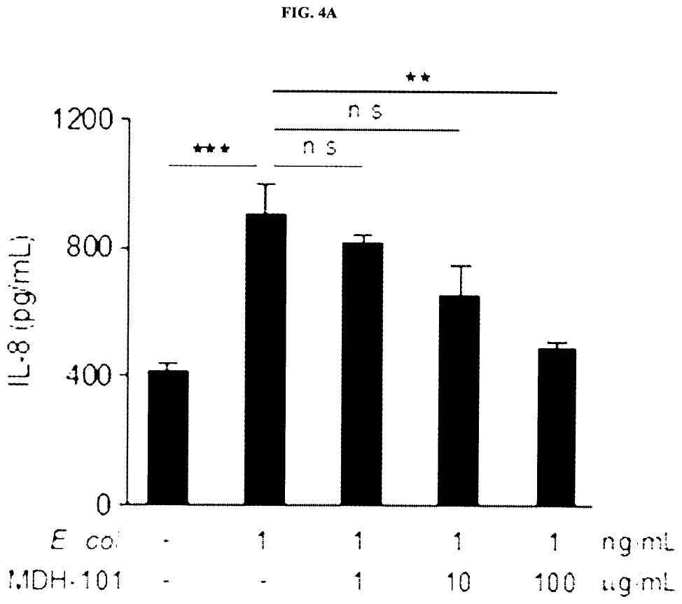

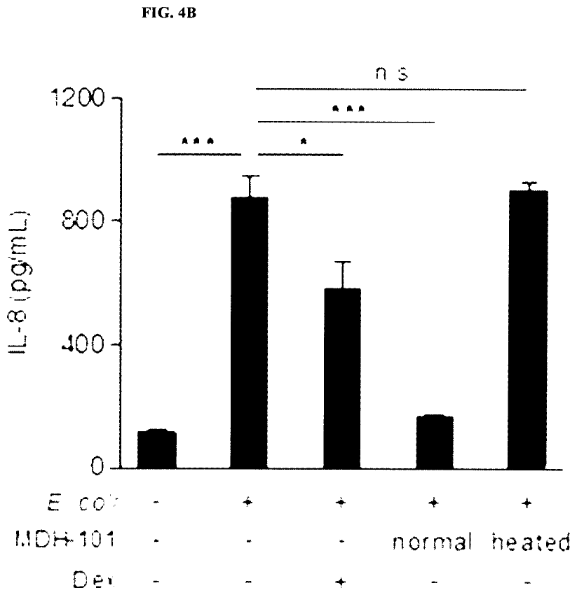

In another exemplary embodiment of the present invention, it was confirmed that the secretion of an inflammatory cytokine (IL-8) was suppressed in a dose-dependent manner when epithelial cells were treated with Micrococcus luteus-derived vesicles (see Example 3 and FIGS. 4A to 4B).

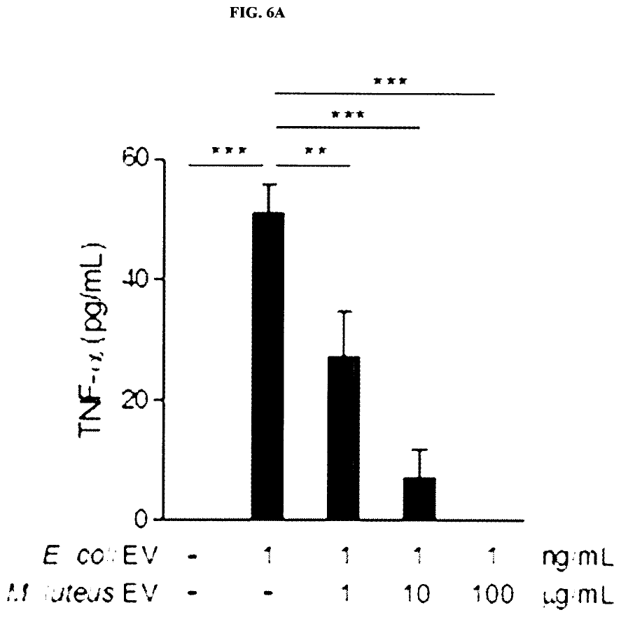

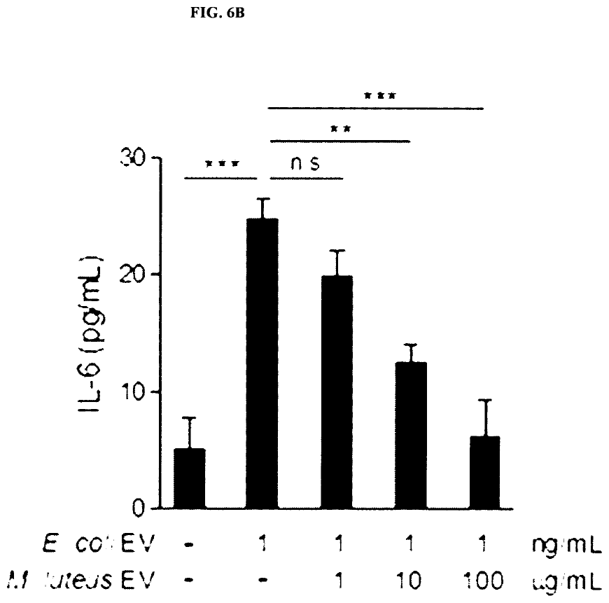

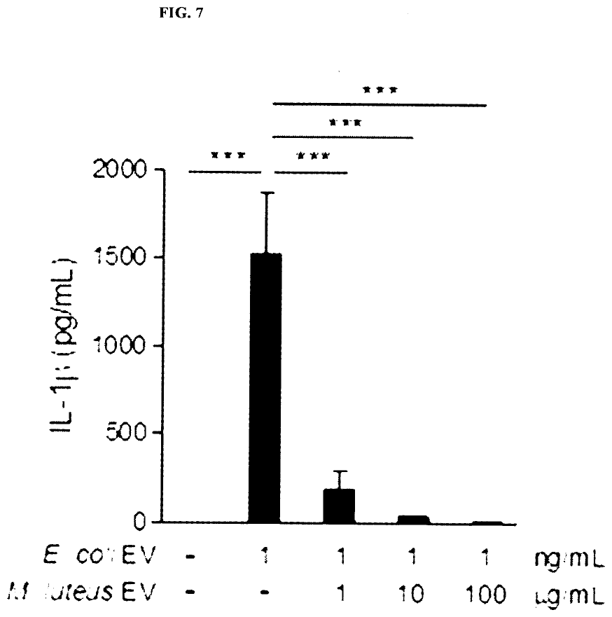

In still another exemplary embodiment of the present invention, it was confirmed that the secretion of inflammatory cytokines (TNF-α, IL-6, and IL-1β) was suppressed in a dose-dependent manner when macrophages were treated with Micrococcus luteus-derived vesicles (see Example 4 and FIGS. 6A to 7).

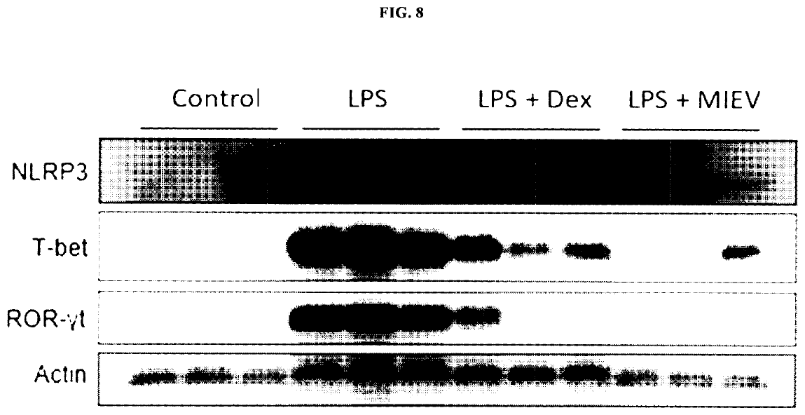

In yet another exemplary embodiment of the present invention, it was confirmed that in LPS-administered mouse models, Micrococcus luteus-derived vesicles efficiently suppressed NLRP3 protein expression, NLRP3inflammasome formation, and innate immune cell (ILC3) generation, which are key signaling components in the pathogenesis of metabolic diseases (see Examples 5 to 7 and FIGS. 8 to 10).

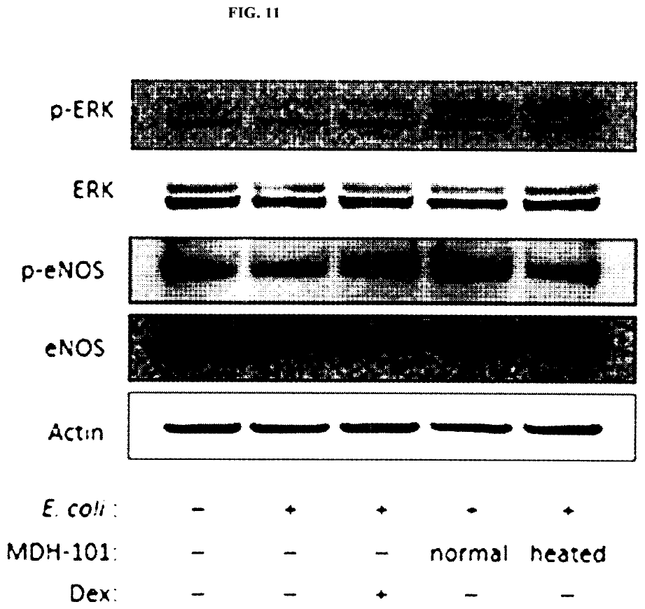

In yet another exemplary embodiment of the present invention, it was confirmed that eNOS signals and ERK signals, which are important signals for cellular homeostasis and vascular health, were suppressed by E. coli-derived vesicles, which are inflammatory causative factors, but were activated by Micrococcus luteus-derived vesicles (see Example 8 and FIG. 11).

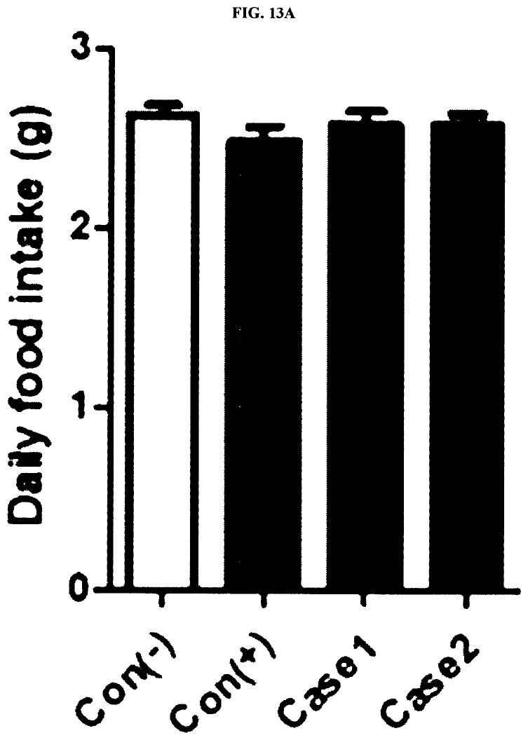

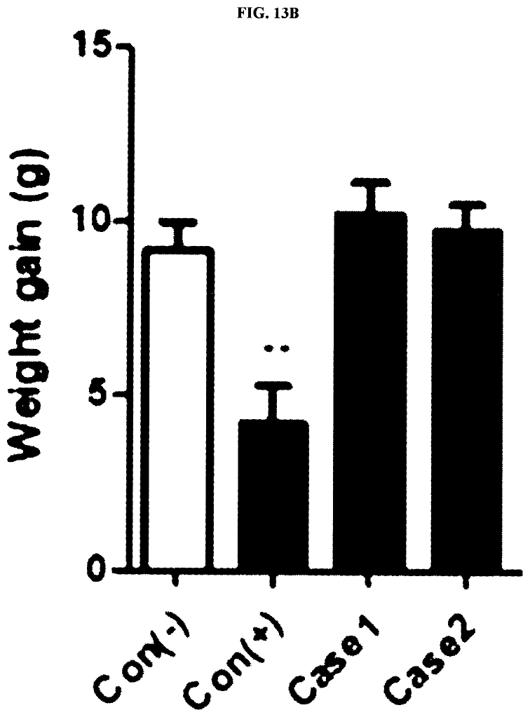

In yet another exemplary embodiment of the present invention, it was confirmed that when Micrococcus luteus-derived vesicles were orally administered to a mouse model of metabolic disease induced by a high-fat diet, body weight was significantly reduced, and the plasma concentrations of triglycerides and free fatty acids, which are biomarkers of metabolic syndrome, and the plasma concentrations of AST and ALT, which are markers of metabolic liver disease, were significantly reduced (see Example 9 and FIGS. 13 to 15).

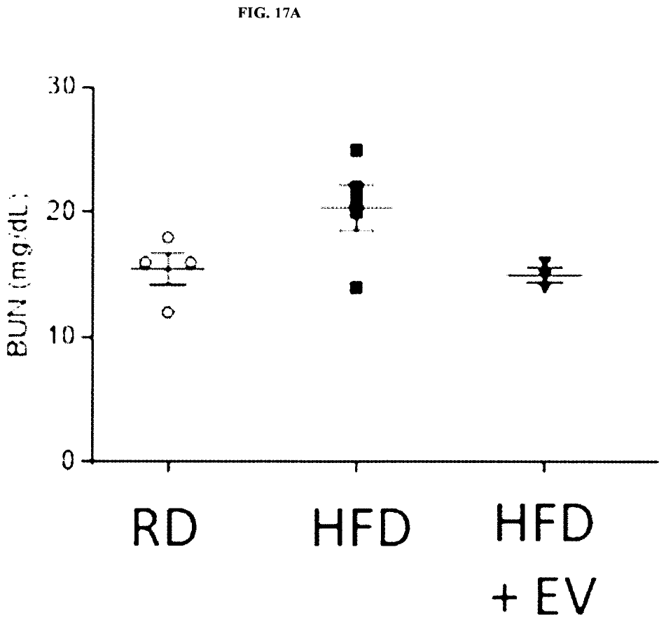

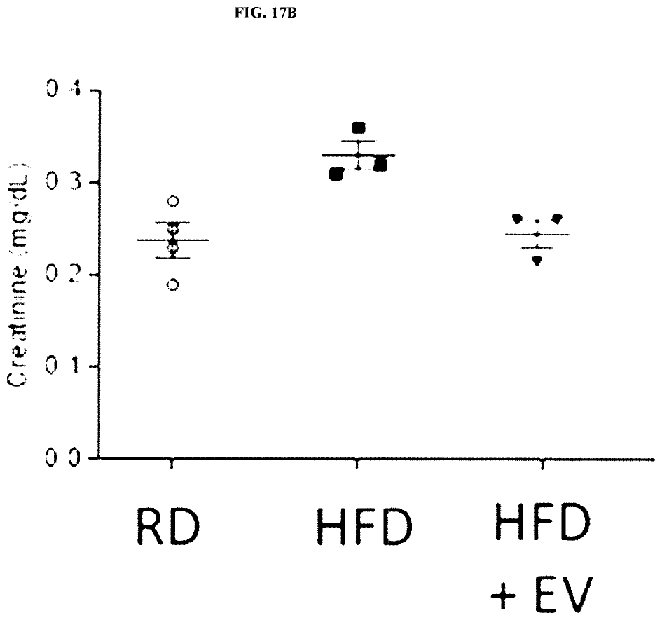

In yet another exemplary embodiment of the present invention, it was confirmed that when Micrococcus luteus-derived vesicles to a mouse model of metabolic renal disease induced by a high-fat diet, the plasma concentrations of BUN and creatinine, which are renal function markers, were reduced to normal levels (see Example 10 and FIGS. 17A and 17B).

Thus, the present invention provide a pharmaceutical composition for preventing, alleviating or treating a metabolic disease, comprising Micrococcus luteus-derived vesicles as an active ingredient.

As used herein, the term “extracellular vesicle” or “vesicle” refers to a structure formed of a nano-sized membrane secreted from various bacteria, and includes, for example, a vesicle derived from gram-negative bacteria such as E. coli, which has, an endotoxin (lipopolysaccharide), a toxic protein, and both bacterial DNA, RNA and like, or a vesicle derived from gram-positive bacteria such as bacteria of the genus Micrococcus, which have outer membrane vesicles (OMVs), a protein and a nucleic acid as well as components of a bacterial cell wall, such as peptidoglycan and lipoteichoic acid.

In the present invention, the extracellular vesicles or vesicles may collectively refer to all structures which are naturally secreted from Micrococcus luteus or consisting of naturally secreted or artificially produced membranes, and may be variously represented by MDH-101, MDH-101EV, M. luteus EV or M1EV in the present invention.

The vesicles may be isolated by heat treatment or autoclaving during Micrococcus luteus culture, or using one or more methods selected from the group consisting of centrifugation, ultracentrifugation, autoclaving, extrusion, sonication, cell lysis, homogenization, freezing-thawing, electroporation, mechanical degradation, chemical treatment, filtration with a filter, gel filtration chromatography, pre-flow electrophoresis, and capillary electrophoresis of the cell culture. In addition, for isolation, washing for removing impurities, and concentration of the obtained vesicles may be further performed.

In the present invention, the vesicles isolated by the method are in the form of spheres, and may have an average diameter of 10 to 200 nm, 10 to 190 nm, 10 to 180 nm, 10 to 170 nm, 10 to 160 nm, 10 to 150 nm, 10 to 140 nm, 10 to 130 nm, 10 to 120 nm, 10 to 110 nm, 10 to 100 nm, 10 to 90 nm, 10 to 80 nm, 10 to 70 nm, 10 to 60 nm, 10 to 50 nm, 20 to 200 nm, 20 to 180 nm, 20 to 160 nm, 20 to 140 nm, 20 to 120 nm, 20 to 100 nm, or 20 to 80 nm, preferably 20 to 200 nm, but the average diameter is not limited thereto.

As used herein, the phrase “comprising as an active ingredient” refers to comprising a sufficient amount to achieve the efficacy or activity of the Micrococcus luteus-derived vesicles.

As used herein, the term “metabolic disease” refers to a disease caused by aging or death of cells caused by inflammation or metabolic stress caused by pathogenic metabolites produced in the body, and may include, for example, a metabolic cardiovascular disease, a metabolic liver disease, a metabolic renal disease, a metabolic musculoskeletal disease, and the like caused by a high-fat diet, and specifically, the metabolic disease may be a metabolic disease mediated by an NLR family pyrin domain containing 3 inflammasome (NLRP3 inflammasome).

As used herein, the phrase “metabolic disease mediated by an NLR family pyrin domain containing 3 inflammasome (NLRP3 inflammasome) refers to a metabolic disease caused by excessive formation of an inflammasome, and in the present invention, the Micrococcus luteus-derived vesicles can suppress the formation of the NLRP3 inflammasome by suppressing NLRP3 protein expression, thereby effectively preventing, ameliorating, or treating metabolic diseases mediated by an NLR family pyrin domain containing 3 inflammasome (NLRP3 inflammasome).

As used herein, the term “metabolic cardiovascular disease” refers to a disease caused by metabolic imbalance of carbohydrates, lipids, and the like in vivo, and may include, for example, hyperinsulinemia, dyslipidemia, arrhythmia, metabolic syndrome, stroke, cardiac failure, and the like, but is not limited thereto.

As used herein, the term “metabolic liver disease” refers to a disease caused by metabolic dysregulation in the liver unrelated to alcohol consumption, and may include hepatic triglyceride accumulation, simple steatosis, non-alcoholic steatohepatitis, hepatocirrhosis, and the like, but is not limited thereto.

As used herein, the term “metabolic renal disease” refers to a disease caused by reduced function of the kidneys to remove metabolic waste products from the blood, and may include glomerulonephritis, diabetic nephropathy, hypertensive nephropathy, chronic renal failure, and the like.

As used herein, the term “metabolic musculoskeletal disease” refers to a disease caused by abnormalities in muscle metabolism and/or bone metabolism, and may include cachexia, gout, sarcopenia, osteoporosis, and the like, but is not limited thereto.

The amount of the vesicles in the composition of the present invention may be appropriately adjusted depending on the symptoms of a disease, the degree of progression of symptoms, the condition of a patient, and the like, and may range from, for example, 0.0001 wt % to 99.9 wt % or 0.001 wt % to 50 wt % with respect to a total weight of the composition, but the present invention is not limited thereto. The amount ratio is a value based on the amount of dried product from which a solvent is removed.

The pharmaceutical composition according to the present invention may further include a suitable carrier, excipient, and diluent which are commonly used in the preparation of pharmaceutical compositions. The excipient may be, for example, one or more selected from the group consisting of a diluent, a binder, a disintegrant, a lubricant, an adsorbent, a humectant, a film-coating material, and a controlled release additive.

The pharmaceutical composition according to the present invention may be used by being formulated, according to commonly used methods, into a form such as powders, granules, sustained-release-type granules, enteric granules, liquids, eye drops, elixirs, emulsions, suspensions, spirits, troches, aromatic water, lemonades, tablets, sustained-release-type tablets, enteric tablets, sublingual tablets, hard capsules, soft capsules, sustained-release-type capsules, enteric capsules, pills, tinctures, soft extracts, dry extracts, fluid extracts, injections, capsules, perfusates, or a preparation for external use, such as plasters, lotions, pastes, sprays, inhalants, patches, sterile injectable solutions, or aerosols. The preparation for external use may have a formulation such as creams, gels, patches, sprays, ointments, plasters, lotions, liniments, pastes, or cataplasmas.

As the carrier, the excipient, and the diluent that may be included in the pharmaceutical composition according to the present invention, lactose, dextrose, sucrose, oligosaccharides, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil may be used.

For formulation, commonly used diluents or excipients such as fillers, thickeners, binders, wetting agents, disintegrants, and surfactants are used.

As additives of tablets, powders, granules, capsules, pills, and troches according to the present invention, excipients such as corn starch, potato starch, wheat starch, lactose, white sugar, glucose, fructose, D-mannitol, precipitated calcium carbonate, synthetic aluminum silicate, dibasic calcium phosphate, calcium sulfate, sodium chloride, sodium hydrogen carbonate, purified lanolin, microcrystalline cellulose, dextrin, sodium alginate, methyl cellulose, sodium carboxymethylcellulose, kaolin, urea, colloidal silica gel, hydroxypropyl starch, hydroxypropyl methylcellulose (HPMC), HPMC 1928, HPMC 2208, HPMC 2906, HPMC 2910, propylene glycol, casein, calcium lactate, and Primojel®; and binders such as gelatin, Arabic gum, ethanol, agar powder, cellulose acetate phthalate, carboxymethylcellulose, calcium carboxymethylcellulose, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, purified shellac, starch, hydroxypropyl cellulose, hydroxypropyl methylcellulose, polyvinyl alcohol, and polyvinylpyrrolidone may be used, and disintegrants such as hydroxypropyl methylcellulose, corn starch, agar powder, methylcellulose, bentonite, hydroxypropyl starch, sodium carboxymethylcellulose, sodium alginate, calcium carboxymethylcellulose, calcium citrate, sodium lauryl sulfate, silicic anhydride, 1-hydroxypropylcellulose, dextran, ion-exchange resin, polyvinyl acetate, formaldehyde-treated casein and gelatin, alginic acid, amylose, guar gum, sodium bicarbonate, polyvinylpyrrolidone, calcium phosphate, gelled starch, Arabic gum, amylopectin, pectin, sodium polyphosphate, ethyl cellulose, white sugar, magnesium aluminum silicate, a di-sorbitol solution, and light anhydrous silicic acid; and lubricants such as calcium stearate, magnesium stearate, stearic acid, hydrogenated vegetable oil, talc, lycopodium powder, kaolin, Vaseline, sodium stearate, cacao butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, Macrogol, synthetic aluminum silicate, silicic anhydride, higher fatty acids, higher alcohols, silicone oil, paraffin oil, polyethylene glycol fatty acid ether, starch, sodium chloride, sodium acetate, sodium oleate, dl-leucine, and light anhydrous silicic acid may be used.

As additives of liquids according to the present invention, water, dilute hydrochloric acid, dilute sulfuric acid, sodium citrate, monostearic acid sucrose, polyoxyethylene sorbitol fatty acid esters (twin esters), polyoxyethylene monoalkyl ethers, lanolin ethers, lanolin esters, acetic acid, hydrochloric acid, ammonia water, ammonium carbonate, potassium hydroxide, sodium hydroxide, prolamine, polyvinylpyrrolidone, ethylcellulose, and sodium carboxymethylcellulose may be used.

In syrups according to the present invention, a white sugar solution, other sugars or sweeteners, and the like may be used, and as necessary, a fragrance, a colorant, a preservative, a stabilizer, a suspending agent, an emulsifier, a viscous agent, or the like may be used.

In emulsions according to the present invention, purified water may be used, and as necessary, an emulsifier, a preservative, a stabilizer, a fragrance, or the like may be used.

In suspensions according to the present invention, suspending agents such as acacia, tragacanth, methylcellulose, carboxymethylcellulose, sodium carboxymethylcellulose, microcrystalline cellulose, sodium alginate, hydroxypropyl methylcellulose (HPMC), HPMC 1828, HPMC 2906, HPMC 2910, and the like may be used, and as necessary, a surfactant, a preservative, a stabilizer, a colorant, and a fragrance may be used.

Injections according to the present invention may include: solvents such as distilled water for injection, a 0.9% sodium chloride solution, Ringer's solution, a dextrose solution, a dextrose+sodium chloride solution, PEG, lactated Ringer's solution, ethanol, propylene glycol, non-volatile oil-sesame oil, cottonseed oil, peanut oil, soybean oil, corn oil, ethyl oleate, isopropyl myristate, and benzene benzoate; cosolvents such as sodium benzoate, sodium salicylate, sodium acetate, urea, urethane, monoethylacetamide, butazolidine, propylene glycol, the Tween series, amide nicotinate, hexamine, and dimethylacetamide; buffers such as weak acids and salts thereof (acetic acid and sodium acetate), weak bases and salts thereof (ammonia and ammonium acetate), organic compounds, proteins, albumin, peptone, and gums; isotonic agents such as sodium chloride; stabilizers such as sodium bisulfite (NaHSO₃) carbon dioxide gas, sodium metabisulfite (Na₂S₂O₅), sodium sulfite (Na₂SO₃), nitrogen gas (N₂), and ethylenediamine tetraacetic acid; sulfating agents such as 0.1% sodium bisulfide, sodium formaldehyde sulfoxylate, thiourea, disodium ethylenediaminetetraacetate, and acetone sodium bisulfite; a pain relief agent such as benzyl alcohol, chlorobutanol, procaine hydrochloride, glucose, and calcium gluconate; and suspending agents such as sodium CMC, sodium alginate, Tween 80, and aluminum monostearate.

In suppositories according to the present invention, bases such as cacao butter, lanolin, Witepsol, polyethylene glycol, glycerogelatin, methylcellulose, carboxymethylcellulose, a mixture of stearic acid and oleic acid, Subanal, cottonseed oil, peanut oil, palm oil, cacao butter+cholesterol, lecithin, lanette wax, glycerol monostearate, Tween or span, imhausen, monolan(propylene glycol monostearate), glycerin, Adeps solidus, buytyrum Tego-G, cebes Pharma 16, hexalide base 95, cotomar, Hydrokote SP, S-70-XXA, S-70-XX75(S-70-XX95), Hydrokote 25, Hydrokote 711, idropostal, massa estrarium (A, AS, B, C, D, E, I, T), masa-MF, masupol, masupol-15, neosuppostal-N, paramount-B, supposiro OSI, OSIX, A, B, C, D, H, L, suppository base IV types AB, B, A, BC, BBG, E, BGF, C, D, 299, suppostal N, Es, Wecoby W, R, S, M, Fs, and tegester triglyceride matter (TG-95, MA, 57) may be used.

Solid preparations for oral administration include tablets, pills, powders, granules, capsules, and the like, and such solid preparations are formulated by mixing the composition with at least one excipient, e.g., starch, calcium carbonate, sucrose, lactose, gelatin, and the like. In addition to simple excipients, lubricants such as magnesium stearate and talc are also used.

Examples of liquid preparations for oral administration include suspensions, liquids for internal use, emulsions, syrups, and the like, and these liquid preparations may include, in addition to simple commonly used diluents, such as water and liquid paraffin, various types of excipients, for example, a wetting agent, a sweetener, a fragrance, a preservative, and the like. Preparations for parenteral administration include an aqueous sterile solution, a non-aqueous solvent, a suspension, an emulsion, a freeze-dried preparation, and a suppository. Non-limiting examples of the non-aqueous solvent and the suspension include propylene glycol, polyethylene glycol, a vegetable oil such as olive oil, and an injectable ester such as ethyl oleate.

The pharmaceutical composition according to the present invention is administered in a pharmaceutically effective amount. In the present invention, “the pharmaceutically effective amount” refers to an amount sufficient to treat diseases at a reasonable benefit/risk ratio applicable to medical treatment, and an effective dosage level may be determined according to factors including types of diseases of patients, the severity of disease, the activity of drugs, sensitivity to drugs, administration time, administration route, excretion rate, treatment period, and simultaneously used drugs, and factors well known in other medical fields.

The composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this may be easily determined by those of ordinary skill in the art.

The pharmaceutical composition of the present invention may be administered to a subject via various routes. All administration methods can be predicted, and the pharmaceutical composition may be administered via, for example, oral administration, subcutaneous injection, intravenous injection, intramuscular injection, intrathecal (space around the spinal cord) injection, sublingual administration, administration via the buccal mucosa, intrarectal insertion, intravaginal insertion, ocular administration, intra-aural administration, intranasal administration, inhalation, spraying via the mouth or nose, transdermal administration, percutaneous administration, or the like.

The pharmaceutical composition of the present invention is determined depending on the type of a drug, which is an active ingredient, along with various related factors such as a disease to be treated, administration route, the age, gender, and body weight of a patient, and the severity of diseases. Specifically, the effective amount of the composition according to the present invention may vary depending on the patient's age, sex, and body weight, and generally, 0.001 to 150 mg of the composition and preferably, 0.01 to 100 mg of the composition, per 1 kg of the body weight, may be administered daily or every other day or may be administered once to three times a day. However, since the effective amount may be increased or decreased depending on the administration route, the severity of obesity, gender, body weight, age, and the like, the dosage is not intended to limit the scope of the present invention in any way.

Further, the present invention provides a method for preventing or treating a metabolic disease, the method comprising administering a composition comprising Micrococcus luteus-derived vesicles as an active ingredient to a subject in need thereof.

In addition, the present invention provides a use of a composition comprising Micrococcus luteus-derived vesicles as an active ingredient for preventing or treating a metabolic disease.

Furthermore, the present invention provides a use of Micrococcus luteus-derived vesicles for preparing a drug for treating a metabolic disease.

As used herein, the “subject” refers to a subject in need of treatment of a disease, and more specifically, refers to a mammal such as a human or a non-human primate, a mouse, a rat, a dog, a cat, a horse, and a cow, but the present invention is not limited thereto.

As used herein, the “administration” refers to providing a subject with a predetermined composition of the present invention by using an arbitrary appropriate method.

The term “prevention” as used herein means all actions that inhibit or delay the onset of a target disease. The term “treatment” as used herein means all actions that alleviate or beneficially change a target disease and abnormal metabolic symptoms caused thereby via administration of the pharmaceutical composition according to the present invention. The term “alleviation” as used herein means all actions that reduce the degree of parameters related to a target disease, e.g., symptoms via administration of the composition according to the present invention.

In addition, the present invention provides a food composition for preventing or alleviating a metabolic disease, comprising Micrococcus luteus-derived vesicles as an active ingredient.

The food composition may be a health functional food composition, but is not limited thereto.

The vesicles according to the present invention may be used by adding an active ingredient as is to food or may be used together with other foods or food ingredients, but may be appropriately used according to a typical method. The mixed amount of the active ingredient may be suitably determined depending on the purpose of use thereof (for prevention or alleviation). In general, when a food or beverage is prepared, the composition of the present invention is added in an amount of 15 wt % or less, preferably 10 wt % or less based on the raw materials. However, for long-term intake for the purpose of health and hygiene or for the purpose of health control, the amount may be less than the above-mentioned range, and the vesicles have no problem in terms of stability, so the active ingredient may be used in an amount more than the above-mentioned range.

The type of food is not particularly limited. Examples of food to which the material may be added include meats, sausage, bread, chocolate, candies, snacks, confectioneries, pizza, instant noodles, other noodles, gums, dairy products including ice creams, various soups, beverages, tea, drinks, alcoholic beverages, vitamin complexes, and the like, and include all health functional foods in a typical sense.

The health beverage composition according to the present invention may contain various flavors or natural carbohydrates, and the like as additional ingredients as in a typical beverage. The above-described natural carbohydrates may be monosaccharides such as glucose and fructose, disaccharides such as maltose and sucrose, polysaccharides such as dextrin and cyclodextrin, and sugar alcohols such as xylitol, sorbitol, and erythritol. As a sweetener, it is possible to use a natural sweetener such as thaumatin and stevia extract, a synthetic sweetener such as saccharin and aspartame, and the like. The proportion of the natural carbohydrates is generally about 0.01 to 0.20 g, or about 0.04 to 0.10 g per 100 ml of the composition of the present invention.

In addition to the aforementioned ingredients, the composition of the present invention may contain various nutrients, vitamins, electrolytes, flavors, colorants, pectic acids and salts thereof, alginic acid and salts thereof, organic acids, protective colloid thickeners, pH adjusters, stabilizers, preservatives, glycerin, alcohols, carbonating agents used in carbonated drinks, and the like. In addition, the composition of the present invention may contain flesh for preparing natural fruit juice, fruit juice drinks, and vegetable drinks. These ingredients may be used either alone or in combinations thereof. The proportion of these additives is not significantly important, but is generally selected within a range of 0.01 to 0.20 part by weight per 100 parts by weight of the composition of the present invention.

In addition, the present invention provides an inhalant composition for preventing or alleviating a metabolic disease, comprising Micrococcus luteus-derived vesicles as an active ingredient.

In the case of an inhalant composition, the compound may be formulated according to a method known in the art, and may be conveniently delivered in the form of an aerosol spray from a pressurized pack or a nebulizer by using a suitable propellant, for example, dichlorofluoromethane, trichlorofluoromethane, dichlorotetrafluoroethane, carbon dioxide, or other suitable gases. In the case of the pressurized aerosol, a dosage unit may be determined by providing a valve for transferring a metered amount. For example, a gelatin capsule and a cartridge for use in an inhaler or insufflator may be formulated so as to contain a powder mixture of a compound and a suitable powder base such as lactose or starch.

Furthermore, the present invention provides a composition for delivering a therapeutic drug for a liver disease or renal disease, comprising Micrococcus luteus-derived vesicles as an active ingredient.

As used herein, the term “drug delivery” refers to all means or actions which load and deliver a drug such as a protein, an antibody, a polymer compound, a low-molecular weight compound, siRNA, and an oligonucleotide to the vesicle according to the present invention in order to deliver the drug to a specific organ, tissue, cell, or organelle.

In the present invention, the composition for delivering a drug may deliver the drug to one or more organs selected from the group consisting of the stomach, small intestine, large intestine, lungs, liver, kidneys, and brain, and preferably may deliver the drug to the liver or kidneys, but is not limited thereto.

Further, the present invention provides a method for delivering a drug for treating a liver disease or renal disease, the method comprising administering a composition comprising Micrococcus luteus-derived vesicles, which carry a drug for treating a liver disease or renal disease, as an active ingredient to a subject in need thereof.

In addition, the present invention provides a use of a composition comprising Micrococcus luteus-derived vesicles as an active ingredient for delivering a therapeutic drug for a liver disease or renal disease.

### Modes of the Invention

Hereinafter, preferred Examples for helping the understanding of the present invention will be suggested. However, the following Examples are provided only to more easily understand the present invention, and the contents of the present invention are not limited by the following Examples.

### EXAMPLES

### Example 1. Isolation of Vesicles from Micrococcus luteus Culture Fluid

After culturing a Micrococcus luteus strain, vesicles thereof were isolated, analyzed and characterized. Micrococcus luteus was cultured in a de Man-Rogosa and Sharpe (MRS) medium until absorbance (OD 600) became 1.0 to 1.5 in a 37° C. aerobic chamber, and then sub-cultured. Subsequently, the medium supernatant containing the strain was recovered, centrifuged at 10,000 g and 4° C. for 20 minutes, and the strain was removed and then filtered through a 0.22-μm filter. And the filtered supernatant was concentrated to a volume of 50 mL using a 100 kDa Pellicon 2 Cassette filter membrane (Merck Millipore, US) and a MasterFlex pump system (Cole-Parmer, US) through microfiltration. Then, the concentrated supernatant was filtered again using a 0.22 μm filter. Subsequently, the protein was quantified using a BCA assay, and the following experiments were performed on the obtained vesicles.

### Example 2. Pharmacokinetic Characteristics of Micrococcus luteus-Derived Vesicles

In order to investigate the pharmacokinetic characteristics of Micrococcus luteus-derived vesicles, the fluorescence expressed in each organ was measured for up to 48 hours by orally administering Micrococcus luteus-derived vesicles stained with a fluorescent staining reagent to mice.

As illustrated in FIG. 1, it was confirmed that when the organ distribution of fluorescently-stained Micrococcus luteus-derived vesicles according to the passage of time was evaluated by images, the vesicles were distributed in various organs. Further, as illustrated in FIG. 2A, it was confirmed that the vesicles were distributed in the liver from 1 hour after oral administration, and the distribution continued up to 72 hours, and as illustrated in FIG. 2B, it was confirmed that the vesicles were distributed in the kidneys from 1 hour after oral administration, and the distribution continued up to 24 hours.

### Example 3. Anti-Inflammatory Effects of Micrococcus luteus-Derived Vesicles in Epithelial Cells

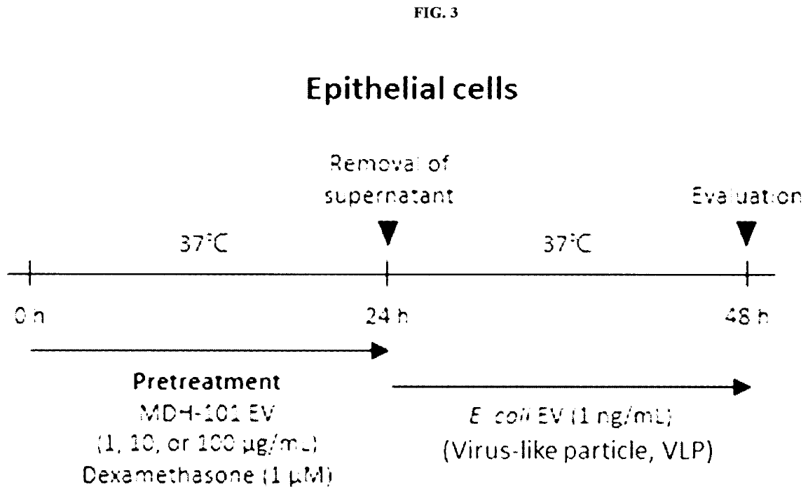

As illustrated in FIG. 3, epithelial cells (A549 cells) were pre-treated with a Micrococcus luteus-derived vesicle (M. luteus EV) or a positive control drug dexamethasone, and then treated with an E. coli-derived vesicle (E. coli EV), which induces inflammation, to measure the secretion amount of an inflammatory cytokine IL-8 by enzyme-linked immunosorbent assay (ELISA, R&D Systems). Specifically, epithelial cells were pre-treated with the Micrococcus luteus-derived vesicle at various concentrations (1, 10, and 100 μg/mL) for 24 hours, and then treated with the E. coli-derived vesicle at a concentration of 1 ng/mL for 24 hours to measure IL-8 secreted into the medium.

As a result, as illustrated in FIG. 4A, it was confirmed that the secretion of IL-8 was suppressed by Micrococcus luteus-derived vesicles in a dose-dependent manner. In addition, as illustrated in FIG. 4B, it was confirmed that when compared to a control drug dexamethasone, the effect of the Micrococcus luteus-derived vesicle on the suppression of IL-8 secretion was even better than the control drug, and when the Micrococcus luteus-derived vesicle was heat-treated, the effect of suppressing the secretion of IL-8 disappeared. From the above results, it can be seen that Micrococcus luteus-derived vesicles have better anti-inflammatory efficacy than the representative anti-inflammatory drug dexamethasone, and from the fact that the anti-inflammatory effect by Micrococcus luteus-derived vesicles is lost upon heat treatment, it can be seen that the anti-inflammatory action is mediated by proteins included in vesicles.

### Example 4. Anti-Inflammatory Effects of Micrococcus luteus-Derived Vesicles in Inflammatory Cells

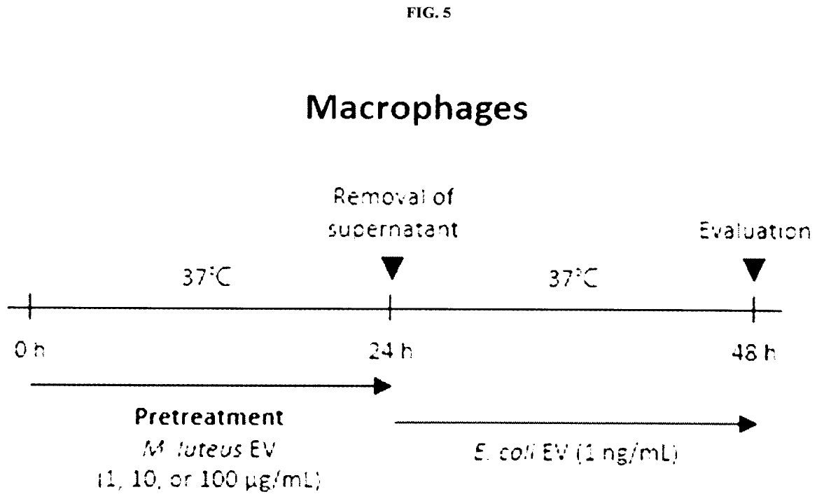

As illustrated in FIG. 5, macrophages were pre-treated with a Micrococcus luteus-derived vesicle (M. luteus EV), and then treated with an E. coli-derived vesicle (E. coli EV), which induces inflammation, to measure the secretion levels of inflammatory cytokines TNF-α, IL-6, and IL-1β by ELISA (R&D Systems). Specifically, after macrophages were pre-treated with the Micrococcus luteus-derived vesicles at various concentrations (1, 10, and 100 μg/mL) for 24 hours, the amounts of TNF-α and IL-6 secreted into the media were measured by treating the macrophages with E. coli-derived vesicles at a concentration of 1 ng/mL for 24 hours.

As a result, as illustrated in FIGS. 6A and 6B, it was confirmed that when macrophages were pre-treated with the Micrococcus luteus-derived vesicle, the secretion of TNF-α (FIG. 6A) and IL-6 (FIG. 6B) by the E. coli-derived vesicle, which is an inflammatory causative factor, was suppressed in a dose-dependent manner by the Micrococcus luteus-derived vesicle (M. luteus EV). This means that Micrococcus luteus-derived vesicles efficiently suppress the secretion of inflammatory mediators by inflammatory causative factors in macrophages, which are representative inflammatory cells that induce inflammation in metabolic diseases.

Further, as illustrated in FIG. 7, it was confirmed that when macrophages were pre-treated with the Micrococcus luteus-derived vesicle, the secretion of IL-1β by the E. coli-derived vesicle (E. coli EV), which is an inflammatory causative factor, was suppressed in a dose-dependent manner by the Micrococcus luteus-derived vesicle (M. luteus EV). This means that Micrococcus luteus-derived vesicles efficiently suppress the secretion of inflammatory mediators increased by an inflammatory complex (inflammasome) in macrophages, which are representative inflammatory cells that induce inflammation in metabolic diseases.

### Example 5. Immune Function-Regulating Effect of Micrococcus luteus-Derived Vesicles in Animal Models

Immune responses to various metabolic stresses are known to be very important for the pathogenesis of metabolic diseases. In particular, the NLRP3 protein present in the cytoplasm is known to be a key signaling pathway in the pathogenesis of metabolic diseases. In order to evaluate the effect of regulating immune function by the Micrococcus luteus-derived vesicles, the expression of NLR family pyrin domain containing 3 (NLRP3), t-box protein expressed in T cells (t-bet), and retineic-acid-receptor-related orphan nuclear receptor gamma (ROR-γt) was confirmed in tissue by western blotting by administering lipopolysaccharide (LPS), which is a representative causative factor inducing immune dysfunction in mice. 50 μg of protein was used to measure the expression amount of each protein, and expression of the above proteins was evaluated in the tissues of a mouse group administered dexamethasone (Dex) or the Micrococcus luteus-derived vesicle.

As a result, as illustrated in FIG. 8, it was confirmed that in a group (LPS) administered LPS, the expression of NLRP3 was remarkably increased compared to that of the negative control, and in tissue of a group (LPS+M1EV) in which the Micrococcus luteus-derived vesicles were administered to the mice administered LPS, the expression of NLRP3 was remarkably suppressed similarly to a group (LPS+Dex) administered dexamethasone. Further, in the group (LPS) administered LPS, the expression of t-bet and ROR-γt proteins was remarkably increased compared to the negative control, and in the group administered the Micrococcus luteus-derived vesicles, the expression of t-bet and ROR-γt proteins was more remarkably suppressed compared to the group administered dexamethasone. This means that the Micrococcus luteus-derived vesicles efficiently suppress innate immune dysfunction caused by an inflammatory causative factor.

### Example 6. Innate Immune Function-Regulating Effect of Micrococcus luteus-Derived Vesicles in Animal Models

Innate immune dysfunction against various metabolic stresses is known to be very important for the pathogenesis of metabolic diseases. It has recently been clarified that Th1 and Th17 acquired immune responses to specific antigens as the pathogenesis of immune diseases are essential for immune dysfunction whereas in the case of metabolic diseases, a metabolite, which acts as a risk factor (danger signal) that induces innate immunity, forms the NLRP3 inflammasome to cause a disease. That is, metabolites such as fatty acids and uric acid act as danger signals to form the NLRP3 inflammasome, but for that purpose, priming processes in which NLPR3 protein expression is induced by inflammatory factors such as LPS and TNF-α are essential.

In order to evaluate the effect of the Micrococcus luteus-derived vesicles on the priming process for NLRP3 inflammasome formation, the degree of activation of JNK and NF-kB (p65), which are signals associated with innate immune function, was evaluated by western blotting by administering LPS to mice by the method in Example 5. 50 μg of protein was used to measure the expression amount of each protein, and expression of the above-described proteins was evaluated in the tissues of a mouse group administered dexamethasone (Dex) or the Micrococcus luteus-derived vesicle.

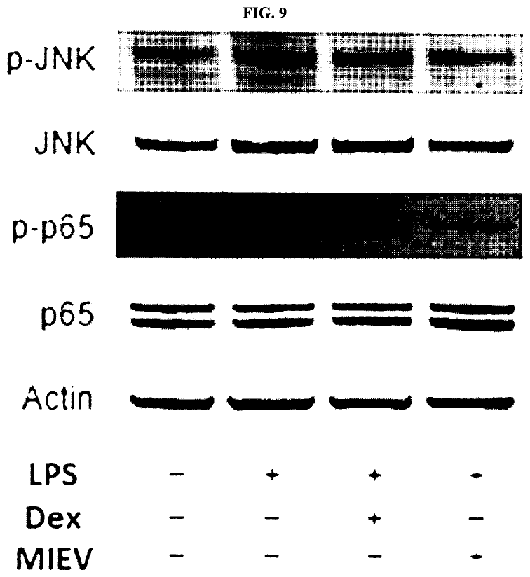

As a result, as illustrated in FIG. 9, in the group administered LPS, phosphorylation of JNK and p65 proteins was induced by LPS compared to the negative control, which was suppressed by dexamethasone (Dex) and the Micrococcus luteus-derived vesicle (MIEV). This means that the Micrococcus luteus-derived vesicles regulate innate immune dysfunction by efficiently suppressing the signaling pathway of JNK and NF-kB (p65) priming NLRP3 inflammasome formation.

### Example 7. Effect of Micrococcus luteus-Derived Vesicles on Innate Immune Cell Generation in Animal Models

It has recently been revealed that ILC3 immune cells through ROR-γt signaling are important in the pathogenesis of a metabolic disease by innate immune dysfunction against various stresses, and the cells secrete IL-17 cytokines and the like to cause a disease. In order to evaluate the effect of the Micrococcus luteus-derived vesicles on the production of ILC3 immune cells, the number of immune cells in tissue was evaluated by a flow cytometry method by administering LPS to mice by the method in Example 5.

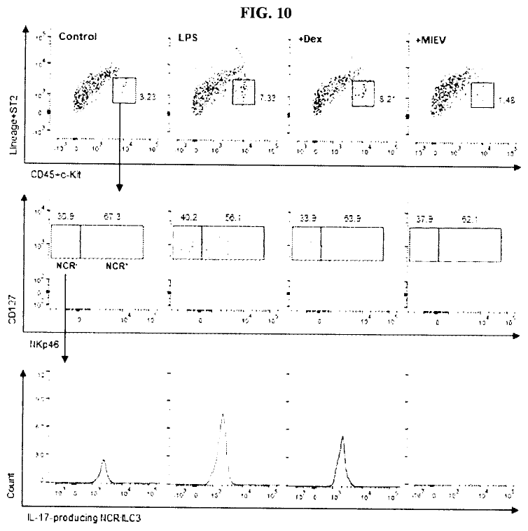

As a result, as illustrated in FIG. 10, ILC3 cells secreting IL-17 were remarkably increased in the group administered LPS compared to the negative control. It was confirmed that the number of ILC3 cells increased by LPS was suppressed by dexamethasone (Dex) and the Micrococcus luteus-derived vesicle (MIEV), and the administration of Micrococcus luteus-derived vesicles suppressed the production of ILC3 cells more remarkably than the administration of dexamethasone. This means that the production of innate immune cells induced by the NLRP3 inflammasome is efficiently suppressed by the Micrococcus luteus-derived vesicle.

### Example 8. Efficacy of Micrococcus luteus-Derived Vesicles on Regulation of Cell Homeostasis Against Oxidative Stress

When cells are repeatedly exposed to various metabolic stresses, this is converted into oxidative stress within the cells to cause cellular senescence and death, leading to a metabolic disease. In the pathogenesis of metabolic diseases, a low concentration of NO nitric oxide (NO) produced through eNOS signals suppresses cell death by antagonizing the action of reactive oxygen species (ROS), which is mainly responsible for oxidative stress, to maintain cell homeostasis. In addition, NO produced through eNOS signals in vascular endothelial cells is well known as an important material that suppresses metabolic vascular diseases.

In order to evaluate the effect of the Micrococcus luteus-derived vesicles on cell homeostasis against oxidative stress, cells were treated with the Micrococcus luteus-derived vesicles by the method described in Example 3, and then the degree of activation of eNOS signals was evaluated. As a method for evaluating the expression of signaling proteins, cells were lysed using a lysis buffer, proteins were extracted, and proteins were quantified using a BCA protein assay kit (Thermo, USA). The degree of protein activation was evaluated using antibodies specific for p-ERK, ERK, p-eNOS, eNOS, and β-actin.

As a result, as illustrated in FIG. 11, upon treatment with the Micrococcus luteus-derived vesicle, the phosphorylation of ERK and eNOS was suppressed by the E. coli-derived vesicle (E. coli), which is an inflammatory factor, but was increased by dexamethasone (Dex) and the Micrococcus luteus-derived vesicle (MDH-101). Further, the activation of eNOS by the Micrococcus luteus-derived vesicle was not induced when the vesicle was heat-treated. From the foregoing results, it can be seen that the Micrococcus luteus-derived vesicles regulate the pathogenesis of a metabolic disease by activating eNOS and ERK signals important for cell homeostasis and vascular health.

### Example 9. Therapeutic Effect of Micrococcus luteus-Derived Vesicles in Mouse Model of Metabolic Disease Induced by High-Fat Diet

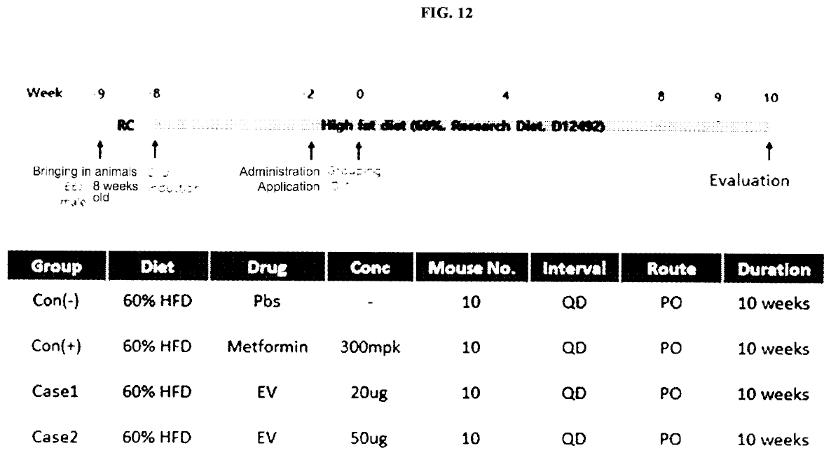

Obesity caused by a high-fat diet and obesity-related metabolic diseases have recently become a major problem. To induce a metabolic disease caused by a high-fat diet, mice were fed a high-fat diet containing 60% saturated fat for 10 weeks, as illustrated in FIG. 12. To evaluate the therapeutic effect of Micrococcus luteus-derived vesicles, the Micrococcus luteus-derived vesicle (EV) was orally administered to mice at 20 μg (low concentration) or 50 μg (high concentration) once daily, metformin was orally administered as a control drug, and PBS was administered in the case of the negative control. The therapeutic effect was evaluated by measuring the plasma concentration of triglyceride (TG) and free fatty acid, which are indicators of metabolic syndrome, and aspartate aminotransferase (AST) and alanine aminotransferase (ALT), which are markers of metabolic liver disease.

As illustrated in FIGS. 13A and 13B, it could be confirmed that food intake (FIG. 13A) and body weight change (FIG. 13B) were not significantly changed in the low-dose vesicle-treated group (Case 1) and high-dose vesicle-treated group (Case 2) compared to the negative control administered PBS, and although there was no difference in food intake in the metformin administration group compared to the PBS administration group, body weight was significantly reduced.

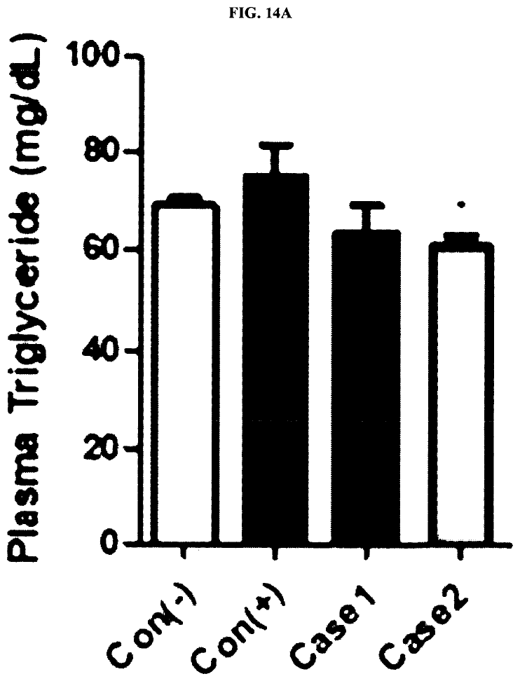

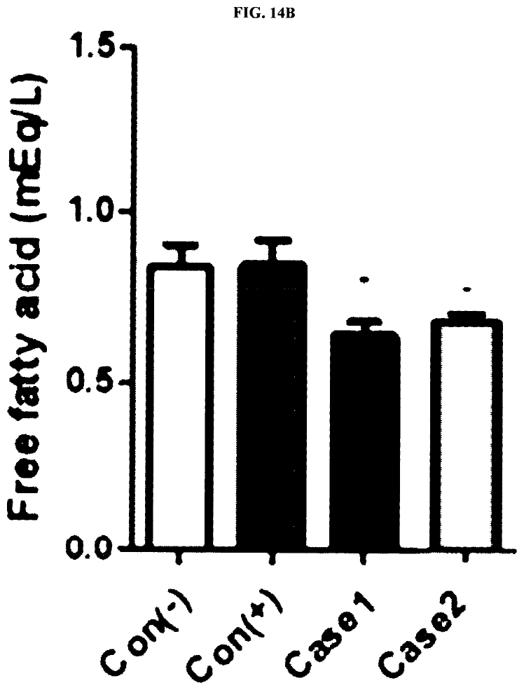

Meanwhile, as illustrated in FIGS. 14A and 14B, it could be confirmed that the concentrations of blood triglycerides (FIG. 14A) and free fatty acids (FIG. 14B), which are indicators of metabolic syndrome, were not decreased by the control drug metformin, but the concentrations were significantly reduced when the vesicles were administered.

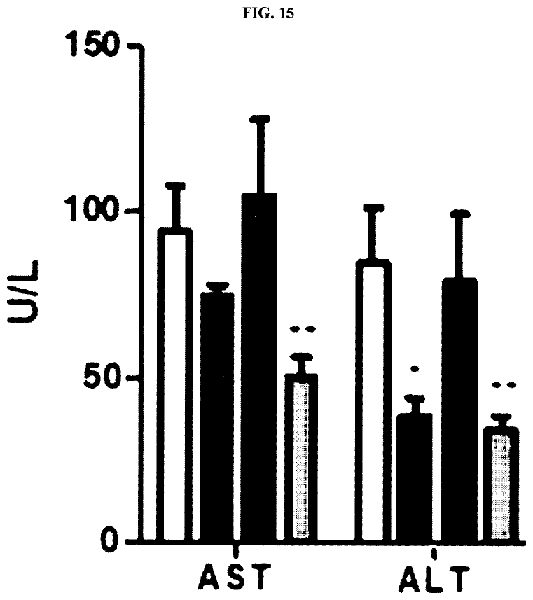

In addition, as illustrated in FIG. 15, similar to the indicator of metabolic syndrome, the plasma AST concentration, which is a marker of a metabolic liver disease such as non-alcoholic steatohepatitis (NASH), was not decreased by metformin, which is a control drug, but significantly decreased when the vesicle was administered, and the plasma ALT concentration was significantly decreased when each of the control drug metformin and the vesicle was administered. This means that Micrococcus luteus-derived vesicles can effectively treat metabolic syndrome and metabolic liver disease induced by a high-fat diet.

### Example 10. Therapeutic Effect of Micrococcus luteus-Derived Vesicles in Mouse Model of Metabolic Renal Disease Induced by High-Fat Diet

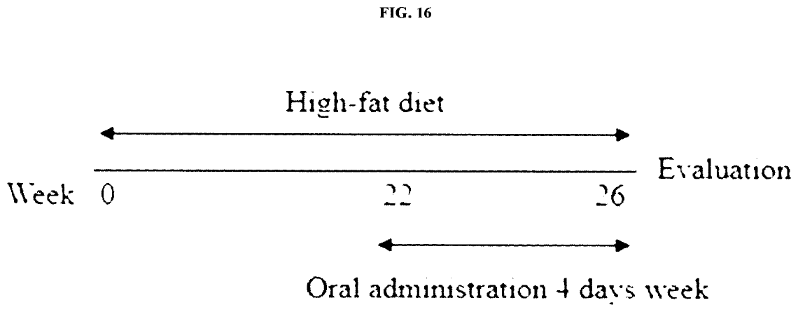

To induce a metabolic renal disease, mice were fed a high-fat diet containing 60% saturated fat for 26 weeks, as illustrated in FIG. 16. To evaluate the therapeutic effect of Micrococcus luteus-derived vesicles, 50 μg of Micrococcus luteus-derived vesicles (EVs) were orally administered to mice once daily for 4 weeks starting from week 22 of administration of a high-fat diet. The therapeutic effect was evaluated by measuring the plasma concentrations of blood urea nitrogen (BUN) and creatinine, which are indicators of renal function.

As a result, as illustrated in FIGS. 17A and 17B, plasma concentrations of BUN and creatinine were increased in mice (HFD) administered a high-fat diet compared to mice (RD) administered a normal diet. In contrast, it could be confirmed that in the case of mice administered the Micrococcus luteus-derived vesicle, plasma concentrations of BUN and creatinine were decreased to the levels of mice fed a normal diet despite the high-fat diet. This means that Micrococcus luteus-derived vesicles can effectively treat a metabolic renal disease such as chronic nephropathy and chronic renal failure.

From the above results, it could be seen that the Micrococcus luteus-derived vesicles of the present invention efficiently suppress the development of a metabolic disease. In particular, it could be seen that the vesicles suppress the formation of the NLRP3 inflammasome, which is a key signaling material in metabolic diseases, to suppress inflammation caused by metabolism disorders, thereby suppressing cell death. Furthermore, it could be seen that the vesicles increase cell homeostasis to suppress cell death by activating the eNOS signals to induce the production of NO, which is a key signaling material for cell homeostasis.

Therefore, the Micrococcus luteus-derived vesicles of the present invention are expected to be able to be used for alleviating, preventing, or treating a metabolic disease.

The above-described description of the present invention is provided for illustrative purposes, and those of ordinary skill in the art to which the present invention pertains will understand that the present invention can be easily modified into other specific forms without changing the technical spirit or essential features of the present invention. Therefore, it should be understood that the above-described Examples are illustrative only in all aspects and are not restrictive.

### INDUSTRIAL APPLICABILITY

The present inventors confirmed that when vesicles derived from Micrococcus luteus were orally administered, the vesicles were absorbed into blood vessels and distributed in organs such as the liver and kidneys. Further, the present inventors confirmed that when epithelial cells and inflammatory cells were treated with the vesicles, the secretion of inflammatory mediators by biological causative factors was remarkably suppressed, and when cells were treated with the vesicles, NLRP3 protein expression and NF-kB signals induced by pathogenic factors were suppressed, and eNOS signals suppressed by pathogenic causative factors were increased. In addition, since it was confirmed that when the vesicles were administered to a mouse model of metabolic liver disease and kidney disease induced by a high-fat diet, hepatitis and renal dysfunction caused by metabolic disorders were efficiently suppressed, it is expected that the Micrococcus luteus-derived vesicles according to the present invention can be usefully used not only for the development of pharmaceuticals or health functional foods for preventing, ameliorating symptoms or treating metabolic cardiovascular diseases such as arteriosclerosis, metabolic syndrome, and cardiac failure; metabolic liver diseases such as non-alcoholic steatohepatitis and hepatocirrhosis; metabolic renal diseases such as chronic nephropathy and renal failure; and metabolic musculoskeletal disorders such as gout, sarcopenia, and osteoporosis, but also as a drug delivery system for treating a liver disease or renal disease.

1.-23. (canceled)

24. A method for treating or alleviating a metabolic disease, the method comprising administering a composition comprising Micrococcus luteus-derived vesicles as an active ingredient to a subject in need thereof.

25. The method of claim 24, wherein the metabolic disease is one or more selected from the group consisting of a metabolic cardiovascular disease, a metabolic liver disease, a metabolic renal disease, and a metabolic musculoskeletal disease.

26. The method of claim 25, wherein the metabolic cardiovascular disease is one or more selected from the group consisting of hyperinsulinemia, dyslipidemia, arrhythmia, metabolic syndrome, arteriosclerosis, stroke, and cardiac failure.

27. The method of claim 25, wherein the metabolic liver disease is one or more selected from the group consisting of hepatic triglyceride accumulation, simple steatosis, non-alcoholic steatohepatitis, and hepatocirrhosis.

28. The method of claim 25, wherein the metabolic renal disease is one or more selected from the group consisting of glomerulonephritis, diabetic nephropathy, hypertensive nephropathy, and chronic renal failure.

29. The method of claim 25, wherein the metabolic musculoskeletal disease is one or more selected from the group consisting of cachexia, gout, sarcopenia, and osteoporosis, but is not limited thereto.

30. The method of claim 24, wherein the metabolic disease is a metabolic disease mediated by an NLR family pyrin domain containing 3 inflammasome (NLRP3 inflammasome).

31. The method of claim 24, wherein the composition suppresses the formation of an NLRP3 inflammasome.

32. The method of claim 24, wherein the vesicles have an average diameter of 10 to 200 nm.

33. The method of claim 24, wherein the vesicles are naturally secreted or artificially produced from Micrococcus luteus.

34. The method of claim 24, wherein the composition is a pharmaceutical composition, a food composition, or an inhalant composition.

35. A method for delivering a drug for treating a liver disease, the method comprising administering a composition comprising Micrococcus luteus-derived vesicles, which carry a drug for treating a liver disease to be targeted, as an active ingredient to a subject in need thereof.

36. A method for delivering a drug for treating a renal disease, the method comprising administering a composition comprising Micrococcus luteus-derived vesicles, which carry a drug for treating a renal disease to be targeted, as an active ingredient to a subject in need thereof.
